(19) United States
(12) Patent Application Publication
(10) Pub. No.: US 2025/0381025 A1
(43) Pub. Date: Dec. 18, 2025

(54) IMPLANTATION DEVICE

(71) Applicant: Vi-Sci LTD, Ramat Gan (IL)

(72) Inventor: Yoni Manor, Tel Aviv (IL)

(21) Appl. No.: 18/878,772

(22) PCT Filed: Jun. 29, 2023
(86) PCT No.: PCT/US2023/069365

§ 371 Date: Dec. 24, 2024

Related U.S. Application Data
(60) Provisional application No. 63/357,307, filed on Jun. 30, 2022.

(51) Int. Cl. A61F 2/16

(52) U.S. Cl. CPC A61F 2/1678

(57) ABSTRACT

A device having a first end, a second end, and a bottom side; wherein the device includes a hollow conical portion at the first end and a hollow cylindrical portion at the second end; wherein the first end is configured to engage an intra-ocular lens injector; wherein the second end includes a pointed tip configured to penetrate tissue of an eye; wherein an opening is formed in the bottom side of the device, extends from the second end toward the first end, is configured to pass therethrough an implantable device received at the first end for implantation in the eye, and is defined by first and second beveled portions extending from the pointed tip toward the first end, first and second parallel portions extending from the beveled portions, first and second concave portions extending from the parallel portions, and a planar face extending from the concave portions.

### CROSS-REFERENCE TO RELATED APPLICATION

This application is an international (PCT) patent application relating to and claiming the benefit of commonly-owned, co-pending U.S. Provisional Patent Application No. 63/357,307, filed on Jun. 30, 2022, and entitled IMPLANTATION DEVICE, the contents of which are incorporated herein by reference in their entirety.

### FIELD OF THE INVENTION

The field of invention relates to a device for use in the implantation of medical devices into tissue. In particular, the field of invention relates to a device for use in the implantation of medical devices into tissue of the human eye.

### BACKGROUND OF THE INVENTION

In the field of ophthalmology, a wide range of illnesses are of chronic nature. Many such illnesses are age related, which consequently present treating physicians with an added layer of complexity. One common challenge in treating eye diseases is patient compliance, i.e. situations in which an effective treatment is available, yet the patient, whether intentionally or not, fails to properly to submit to treatment.

A wide range of attempted solutions to this treatment challenge were and still are in development in order to provide a sustained release-based mechanism of active pharmaceutical ingredients (“APIs”). Some of these solutions include inserts that are positioned in different compartments of the eye (e.g., within the tear canal, intraocularly within a chamber of the eye, or sub-conjunctival). Proper and efficient placement of the devices is critical to the safety of the process and to the success of the treatment. The nature of the placement process also has commercial significance, as patients avoid unpleasant treatments.

Sub-conjunctival placement of an insert often includes a process of “carving a pocket” within tissue of the eye. This process will require forming a snip in the conjunctiva, separation of the conjunctive from underlying tissues, and later closing of the snip. Typically, this is done under local anesthesia, which is both stressful and inconvenient to the patient. Additionally, due to variability in this procedure, at times an insert may not be tightly tucked into place, possibly resulting in dislocation to an extent such that the insert forces its way out of the eye through the conjunctiva and the treatment is ceased.

Delivery systems in the form of intra-ocular lens (“IOL”) injectors are commercially available in different shapes and designs. However, all are characterized with a blunt tip fitted in size to a snip in the cornea, which must be made in advance with a dedicated tool. Such delivery devices are also designed to be used in a specific orientation with respect to the eye, for ergonomic and safety considerations.

### BRIEF DESCRIPTION OF THE FIGURES

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 shows a perspective view of an exemplary implantation device.

FIG. 2 shows a side view of the exemplary implantation device shown in FIG. 1.

FIG. 3 shows a bottom view of the exemplary implantation device shown in FIG. 1.

FIG. 4 shows a front view of the exemplary implantation device shown in FIG. 1.

FIG. 5 shows a detailed bottom view of a tip of the exemplary implantation device shown in FIG. 1.

FIG. 6 shows a detailed front view of a portion of the front view shown in FIG. 4.

FIG. 7 shows a cross-sectional view of the exemplary implantation device shown in FIG. 1, taken at the location labeled 7-7 in FIG. 2.

FIG. 8 shows a detailed view of a portion of the cross-sectional view shown in FIG. 7.

### SUMMARY OF THE INVENTION

In an embodiment, an implantation device has a first end and a second end opposite the first end; the first end is configured to engage an IOL injector; and the second end includes a pointed tip configured to penetrate tissue of an eye and an insertion portion configured to pass therethrough an implantable device from the IOL injector for implantation in the eye.

In some embodiments, a device includes a body having a first end, a second end opposite the first end, a top side extending from the first end to the second end, and a bottom side opposite the top side, wherein the body comprises: a first portion having a hollow and substantially conical shape about a longitudinal axis, the first portion extending from the first end of the body to a transition intermediate the first and second ends of the body, and a second portion having a hollow and substantially cylindrical shape about the longitudinal axis, the second portion extending from the transition to the second end of the body, wherein the first portion and the second portion define a passage having a first inner diameter at the first end and a second inner diameter at the second end, wherein the first inner diameter is larger than the second inner diameter, wherein the first end is configured to engage an intra-ocular lens injector; wherein the second end includes a pointed tip configured to penetrate tissue of an eye, and wherein an insertion opening is formed in the bottom side of the body and extends from the second end of the body toward the first end of the body, wherein the insertion opening is configured to allow to pass therethrough an implantable device received at the first end of the body from the intra-ocular lens injector that is engaged with the first end of the body for implantation of the implantable device in the eye, wherein the insertion opening, when viewed from a lateral side of the body, is defined by: first and second beveled portions extending from the top side of the body toward the longitudinal axis and from the pointed tip toward the first end of the body, wherein each of the first and second beveled portions is concave with respect to the longitudinal axis when viewed from the lateral side of the body, and wherein the first and second beveled portions are positioned within the second portion of the body; first and second parallel portions extending from corresponding ones of the first and second beveled portions in a direction toward the first end of the body, wherein each of the first and second parallel portions is substantially parallel to the longitudinal axis when viewed from the lateral side of the body, and wherein the first and second beveled portions extend across the transition between the first and second portions of the body; first and second concave portions extending from corresponding ones of the first and second parallel portions in a direction toward the first end of the body and toward and past the longitudinal axis of the body, wherein the first and second concave portions are concave with respect to the longitudinal axis when viewed from the lateral side of the body, and wherein the first and second concave portions are positioned within the first portion of the body; and a planar face extending from the first and second concave portions in a direction toward the first end of the body and away from the longitudinal axis of the body, wherein the planar face is angled at a bevel angle that is an acute angle with respect to the longitudinal axis of the body when viewed from the lateral side of the body, and wherein the planar face is positioned within the first portion of the body.

In some embodiments, respective transitions between the first and second concave portions and the planar face are curved.

In some embodiments, the bevel angle is in a range of from 10 degrees to 14 degrees. In some embodiments, the pointed tip is angled with respect to the planar face at a lancet angle that is in a range of from 8 degrees to 12 degrees.

In some embodiments, the first and second concave portions have a radius of curvature that is in a range of from 1 millimeter to 3 millimeters.

In some embodiments, the pointed tip defines a tip angle that is in a range of from 60 degrees to 68 degrees.

In some embodiments, the device is configured for use with an implantable device having an implantable device length, and wherein the insertion opening has a length along the longitudinal axis that is greater than the implantable device length by from 0.5 mm to 2 mm.

In some embodiments, the second portion of the body and the first and second parallel portions define an arc angle that is in a range of from 90 degrees to 150 degrees.

In some embodiments, the second portion of the body has an inner diameter that is in a range of from 1.5 mm to 3 mm.

In some embodiments, the first end of the body has an inner diameter that is in a range of from 2 mm to 5 mm.

In some embodiments, a length of the device along the longitudinal axis is in a range of from 7 mm to 20 mm.

### DETAILED DESCRIPTION OF THE INVENTION

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

As used herein, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.” All ranges used herein are inclusive, i.e., include the stated upper and lower ends thereof as well as all values therebetween.

Unless otherwise defined, all terms (including technical and scientific terms used herein have the same meaning as commonly understood by one having ordinary skill in the art to which this invention belongs. It will further be understood that terms, such as those defined, in commonly used dictionaries, should be interpreted as having a meaning that is consistent with their meaning in the context of the relevant art and the present disclosure and will not be interpreted in an idealized or overly formal sense unless expressly defined.

The present disclosure describes an exemplary embodiment of an add-on insertion device that is configured to couple to standard commercially available IOL delivery systems. It will be apparent to those of skill in the art that the general principles embodied in the exemplary device may also be embodied in other devices.

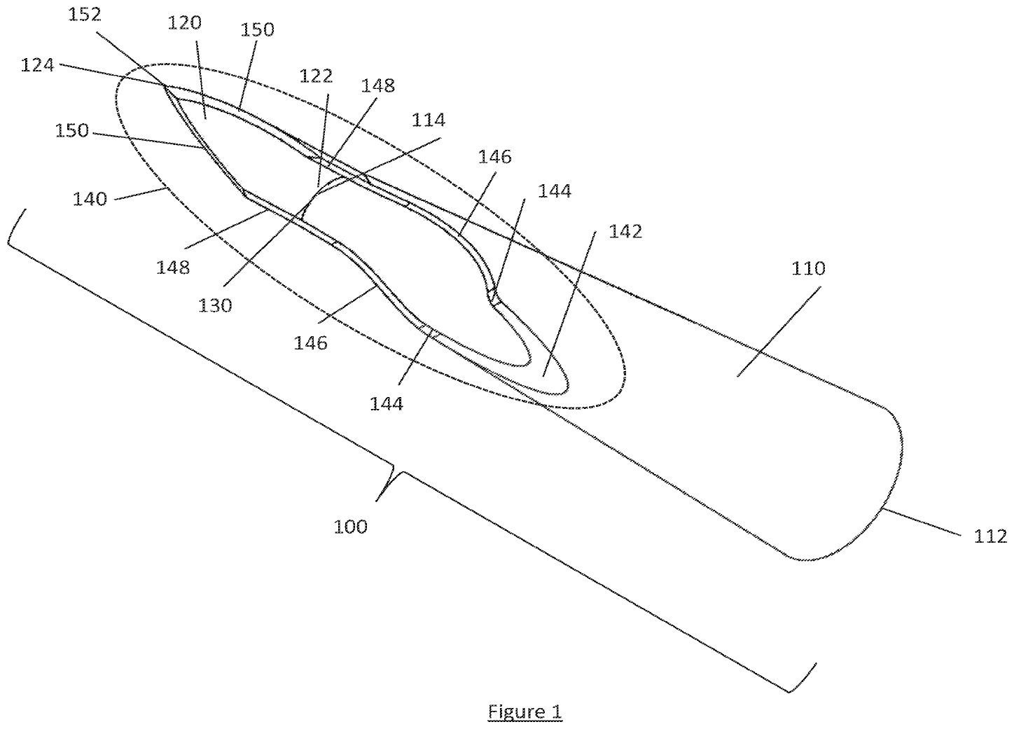

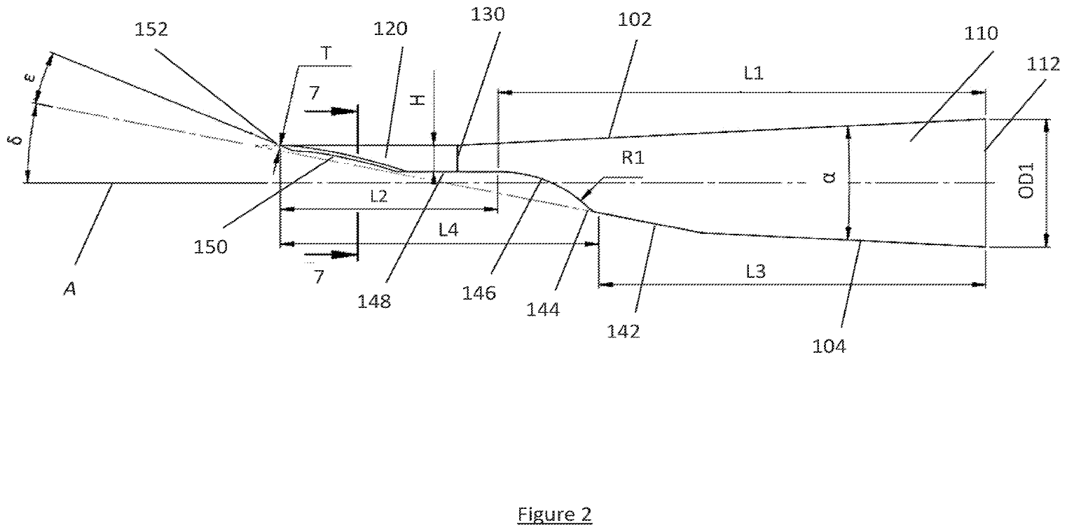

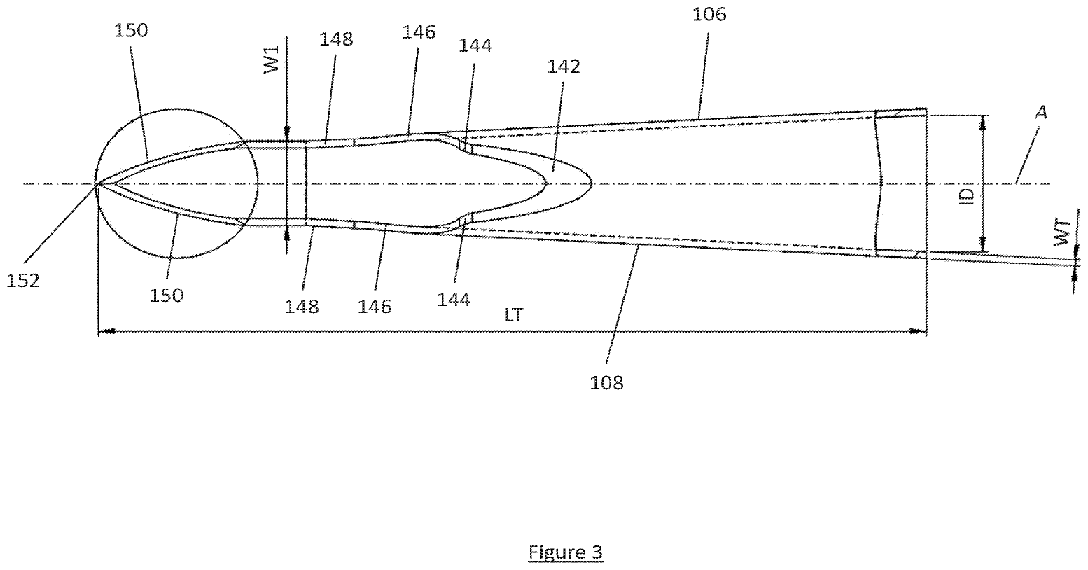

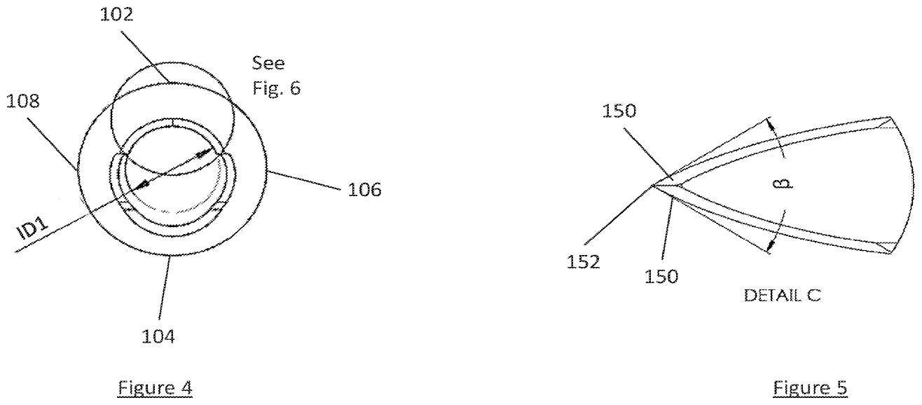

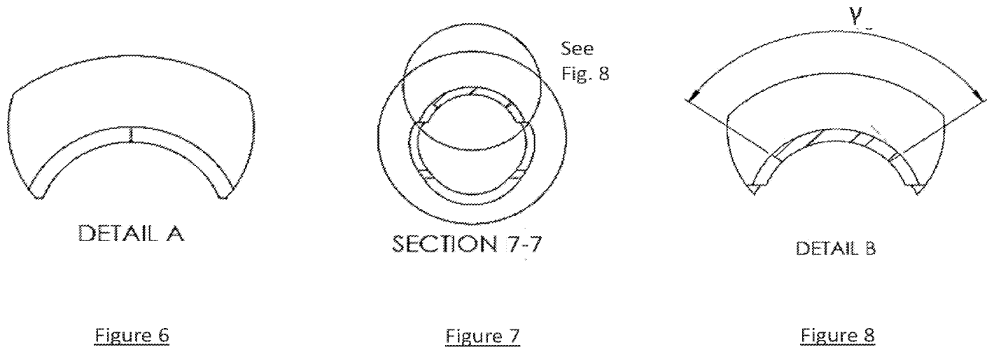

FIG. 1-8 show various views of an exemplary implantation device 100 (for brevity, “device 100”). Certain ones of FIGS. 1-8 include dimensions of certain aspects of the device 100, with lengths shown as measured in millimeters and angles shown as measured in degrees. However, it will be apparent to those of skill in the art that the specific dimensions shown in FIGS. 1-8 are only exemplary and that other dimensions are possible without departing from the general concepts embodied by the exemplary embodiments, including, but not limited to, those described herein. FIG. 1 shows a perspective view of the device 100. FIG. 2 shows a side view of the device 100. FIG. 3 shows a bottom view of the device 100. FIG. 4 shows a front view of the device 100. FIG. 5 shows a detailed bottom view of a tip of the device 100. FIG. 6 shows a detailed front view of a portion of the device 100 as shown in FIG. 4. FIG. 7 shows a cross-sectional view of the device 100 taken at the location labeled 7-7 in FIG. 2. FIG. 8 shows a detailed view of a portion of the cross-sectional view shown in FIG. 7.

Referring now to FIGS. 1-3, the device 100 has a body including a fitting section 110 (i.e., a first portion) and a protrusion 120 (i.e., a second portion) that meet at an arcuate transition 130. The device 100 is oriented about a longitudinal axis A. The fitting section 110 includes a first end 112 that is configured for coupling to an intraocular lens (“IOL”) cartridge and a second end 114 that is opposite the first end 112 and is coincident with the transition 130. In some embodiments, the fitting section 110 has the general shape of a hollow right circular cone about the longitudinal axis A having portions removed therefrom as will be described hereinafter. The protrusion 120 includes a first end 122 that is coincident with the transition 130 and a second end 124 opposite the first end 122. In some embodiments, the protrusion 120 has a hollow and substantially cylindrical shape about the longitudinal axis A having portions removed therefrom as will be described hereinafter. In some embodiments, the hollow interior of the fitting section 110 and the hollow interior of the protrusion 120 cooperate to define a passage.

In some embodiments, the device 100 is formed from a single piece of material (e.g., is a monolithic item). In some embodiments, the device 100 is formed from a material that is biocompatible and has sufficient stiffness to hold a sharp edge. In some embodiments, the material is an alloy. In some embodiments, the material is a steel alloy. In some embodiments, the material is a stainless steel alloy. In some embodiments, the material is 304L stainless steel. In some embodiments, the material is a polymer. In some embodiments, the polymer is polymethyl methacrylate (“PMMA”).

Referring now to FIG. 2, the device 100 includes a top 102 and a bottom 104. Referring now to FIG. 3, the device 100 includes a left side 106 and a right side 108 (e.g., a first lateral side and a second lateral side). It will be apparent to those of skill in the art that the terms “top,” “bottom,” “left,” and “right” are used herein to provide a frame of reference for description of the various elements of the device 100, and that, when in use, the device 100 may be positioned in any orientation with respect to a real-world coordinate system, and need not be oriented such that the top 102, the bottom 104, the left side 106, or the right side 108 are positioned in any particular direction.

Referring now to FIGS. 1-3, the bottom 104 of the device 100 includes an insertion portion 140, which includes certain surfaces that will be described hereinafter. In some embodiments, the insertion portion 140 defines an insertion opening. In some embodiments, such as that shown in FIGS. 1-3, the insertion portion 140 is symmetric (i.e., the two sides of the insertion portion 140 are mirror images of one another to opposite sides of the longitudinal axis A). However, in other embodiments, the insertion portion 140 is not symmetric (i.e., is different to the “left” side of the device than to the “right” side of the device). In some embodiments, the insertion portion 140 includes a planar face 142 formed in the fitting section 110. In some embodiments, the planar face 142 is angled with respect to the longitudinal axis A. In some embodiments, the planar face 142 is angled with respect to the longitudinal axis A by an angle that is from 5 degrees to 45 degrees. In some embodiments, the planar face 142 provides for a smooth progression of the device 100 along tissues of the eye.

Continuing to refer to FIGS. 1-3, in some embodiments, the insertion portion 140 includes first and second convex portions 144 extending from the planar face 142 toward the longitudinal axis A and away from the first end 112 of the fitting section 110. In some embodiments, the convex portions 144 are convex with respect to the longitudinal axis A, as viewed from the side as shown in FIG. 2, with a first one of the convex portions 144 being located to the left side 106 and a second one of the convex portions 144 being located to the right side 108 of the device 100. In some embodiments, the size of the first and second convex portions 144 (e.g., the radius of curvature of the first and second convex portions) is determined as the sizing necessary to provide a smooth transition between the planar face 142 and the first and second concave portions 146. In some embodiments, the radius of curvature of the first and second convex portions 144 is from 0 (e.g., the first and second convex portions 144 are missing and the planar face 142 transitions directly into the first and second concave portions 146) to 3.5 millimeters.

Continuing to refer to FIGS. 1-3, in some embodiments, the insertion portion 140 includes first and second concave portions 146, which extend from respective ones of the first and second convex portions 144 in a direction toward and past the longitudinal axis A, as viewed from the side as shown in FIG. 2, and away from the first end 112 of the fitting section 110. In some embodiments, the first and second concave portions 146 are concave with respect to the bottom 104 of the device 100. In some embodiments, the radius of curvature R1 of the first and second concave portions 146 is from 1 millimeter to 3 millimeters. In some embodiments, the radius of curvature R1 of the first and second concave portions 146 is from 1 millimeter to 2.5 millimeters. In some embodiments, the radius of curvature R1 of the first and second concave portions 146 is from 1 millimeter to 2 millimeters. In some embodiments, the radius of curvature R1 of the first and second concave portions 146 is from 1 millimeter to 1.5 millimeters. In some embodiments, the radius of curvature R1 of the first and second concave portions 146 is from 1.5 millimeters to 3 millimeters. In some embodiments, the radius of curvature R1 of the first and second concave portions 146 is from 1.5 millimeters to 2.5 millimeters. In some embodiments, the radius of curvature R1 of the first and second concave portions 146 is from 1.5 millimeters to 2 millimeters. In some embodiments, the radius of curvature R1 of the first and second concave portions 146 is from 2 millimeters to 3 millimeters. In some embodiments, the radius of curvature R1 of the first and second concave portions 146 is from 2 millimeters to 2.5 millimeters. In some embodiments, the radius of curvature R1 of the first and second concave portions 146 is from 2.5 millimeter to 3 millimeters.

Continuing to refer to FIGS. 1-3, in some embodiments, the insertion portion 140 includes first and second parallel portions 148, which extend from respective ones of the first and second concave portions 146 in a direction away from the first end 112 of the fitting section 110 and parallel to the longitudinal axis A, as viewed from the side as shown in FIG. 2. In some embodiments, the first and second parallel portions 148 are generally parallel to the longitudinal axis A (e.g., angled with respect to the longitudinal axis A by no more than 10 degrees) In some embodiments, the first and second parallel portions extend across the transition 130 from the fitting section 110 to the protrusion 120.

Continuing to refer to FIGS. 1-3, in some embodiments, the insertion portion 140 includes first and second beveled portions 150, which extend from respective ones of the first and second parallel portions 148 in a direction away from the first end 112 of the fitting section 110 and away from the longitudinal axis A, as viewed from the side as shown in FIG. 2. In some embodiments, the first and second beveled portions 150 are concave with respect to the longitudinal axis A.

Continuing to refer to FIGS. 1-3, in some embodiments, the first and second beveled portions 150 meet at a tip 152. In some embodiments, such as shown in FIG. 3, the tip 152 is aligned with the longitudinal axis A when the device 100 is viewed from the bottom, i.e., the tip 152 is centered with respect to the left side 106 and the right side 108. In some embodiments, the tip is 152 is asymmetrical (e.g., is not centered with respect to the left side 106 and the right side 108). Referring now to FIG. 5, in some embodiments, the first and second beveled portions 150 meet at a tip angle β. In some embodiments, the tip angle β is 64.8 degrees. In some embodiments, the tip angle β is between 60 and 70 degrees. In some embodiments, the tip angle β is between 60 and 68 degrees. In some embodiments, the tip angle β is between 60 and 66 degrees. In some embodiments, the tip angle β is between 60 and 64 degrees. In some embodiments, the tip angle β is between 60 and 62 degrees. In some embodiments, the tip angle β is between 62 and 70 degrees. In some embodiments, the tip angle B is between 62 and 68 degrees. In some embodiments, the tip angle β is between 62 and 66 degrees. In some embodiments, the tip angle β is between 62 and 64 degrees. In some embodiments, the tip angle β is between 64 and 70 degrees. In some embodiments, the tip angle β is between 64 and 68 degrees. In some embodiments, the tip angle β is between 64 and 66 degrees. In some embodiments, the tip angle B is between 66 and 70 degrees. In some embodiments, the tip angle B is between 66 and 68 degrees. In some embodiments, the tip angle β is between 68 and 70 degrees. In some embodiments, the tip angle β is between 64.5 and 65.5 degrees.

Referring now to FIG. 2, the fitting section 110 has an outside diameter OD1 at the first end 112 thereof. In some embodiments, the outside diameter OD1 varies based on the size (e.g., diameter) of an implantable device to be implanted through use of the device 100. In some embodiments, the outside diameter OD1 is between 2.0 mm and 5.0 mm. In some embodiments, the outside diameter OD1 is between 2.0 mm and 4.5 mm. In some embodiments, the outside diameter OD1 is between 2.0 mm and 4.0 mm. In some embodiments, the outside diameter OD1 is between 2.0 mm and 3.5 mm. In some embodiments, the outside diameter OD1 is between 2.0 mm and 3.0 mm. In some embodiments, the outside diameter OD1 is between 2.0 mm and 2.5 mm. In some embodiments, the outside diameter OD1 is between 2.5 mm and 5.0 mm. In some embodiments, the outside diameter OD1 is between 2.5 mm and 4.5 mm. In some embodiments, the outside diameter OD1 is between 2.5 mm and 4.0 mm. In some embodiments, the outside diameter OD1 is between 2.5 mm and 3.5 mm. In some embodiments, the outside diameter OD1 is between 2.5 mm and 3.0 mm. In some embodiments, the outside diameter OD1 is between 3.0 mm and 5.0 mm. In some embodiments, the outside diameter OD1 is between 3.0 mm and 4.5 mm. In some embodiments, the outside diameter OD1 is between 3.0 mm and 4.0 mm. In some embodiments, the outside diameter OD1 is between 3.0 mm and 3.5 mm. In some embodiments, the outside diameter OD1 is between 3.5 mm and 5.0 mm. In some embodiments, the outside diameter OD1 is between 3.5 mm and 4.5 mm. In some embodiments, the outside diameter OD1 is between 3.5 mm and 4.0 mm. In some embodiments, the outside diameter OD1 is between 4.0 mm and 5.0 mm. In some embodiments, the outside diameter OD1 is between 4.0 mm and 4.5 mm. In some embodiments, the outside diameter OD1 is between 4.5 mm and 5.0 mm. In some embodiments, the outside diameter OD1 is between 3.0 mm and 3.8 mm. In some embodiments, the outside diameter OD1 is between 3.1 mm and 3.7 mm. In some embodiments, the outside diameter OD1 is between 3.2 mm and 3.6 mm. In some embodiments, the outside diameter OD1 is between 3.3 mm and 3.5 mm. In some embodiments, the outside diameter OD1 is approximately 3.4 millimeters. In some embodiments, the outside diameter OD1 is 3.4 millimeters.

Referring now to FIG. 3, the fitting section 110 has a hollow and substantially conical shape including an inside diameter ID at the first end 112 thereof. In some embodiments, the inside diameter ID, coupled with the conical shape of the fitting section 110, is configured to provide for a press fit with an IOL injector to which the device 100 is to be coupled. In some embodiments, the inside diameter ID may depend on the size of an implantable device to be received at the first end 112 and implanted through use of the device 100. In some embodiments, the inside diameter ID is from 1.8 mm to 4.8 mm. In some embodiments, the inside diameter ID is from 1.8 mm to 4.3 mm. In some embodiments, the inside diameter ID is from 1.8 mm to 3.8 mm. In some embodiments, the inside diameter ID is from 1.8 mm to 3.3 mm. In some embodiments, the inside diameter ID is from 1.8 mm to 2.8 mm. In some embodiments, the inside diameter ID is from 1.8 mm to 2.3 mm. In some embodiments, the inside diameter ID is from 2.3 mm to 4.8 mm. In some embodiments, the inside diameter ID is from 2.3 mm to 4.3 mm. In some embodiments, the inside diameter ID is from 2.3 mm to 3.8 mm. In some embodiments, the inside diameter ID is from 2.3 mm to 3.3 mm. In some embodiments, the inside diameter ID is from 2.3 mm to 2.8 mm. In some embodiments, the inside diameter ID is from 2.8 mm to 4.8 mm. In some embodiments, the inside diameter ID is from 2.8 mm to 4.3 mm. In some embodiments, the inside diameter ID is from 2.8 mm to 3.8 mm. In some embodiments, the inside diameter ID is from 2.8 mm to 3.3 mm. In some embodiments, the inside diameter ID is from 3.3 mm to 4.8 mm. In some embodiments, the inside diameter ID is from 3.3 mm to 4.3 mm. In some embodiments, the inside diameter ID is from 3.3 mm to 3.8 mm. In some embodiments, the inside diameter ID is from 3.8 mm to 4.8 mm. In some embodiments, the inside diameter ID is from 3.8 mm to 4.3 mm. In some embodiments, the inside diameter ID is from 4.3 mm to 4.8 mm. In some embodiments, the inside diameter ID is from 2.8 mm to 3.4 mm. In some embodiments, the inside diameter ID is from 2.9 mm to 3.3 mm. In some embodiments, the inside diameter ID is from 3.0 mm to 3.2 mm. In some embodiments, the inside diameter ID is approximately 3.1 millimeters. In some embodiments, the inside diameter ID is 3.1 millimeters.

Continuing to refer to FIG. 3, the fitting section 110 has a wall thickness WT at the first end thereof. In some embodiments, the wall thickness WT is constant throughout the device 100. In some embodiments, the wall thickness WT varies within the device 100. In some embodiments, the wall thickness WT is from 0.08 mm to 0.2 mm. In some embodiments, the wall thickness WT is from 0.08 mm to 0.17 mm. In some embodiments, the wall thickness WT is from 0.08 mm to 0.14 mm. In some embodiments, the wall thickness WT is from 0.08 mm to 0.11 mm. In some embodiments, the wall thickness WT is from 0.11 mm to 0.2 mm. In some embodiments, the wall thickness WT is from 0.11 mm to 0.17 mm. In some embodiments, the wall thickness WT is from 0.11 mm to 0.14 mm. In some embodiments, the wall thickness WT is from 0.14 mm to 0.2 mm. In some embodiments, the wall thickness WT is from 0.14 mm to 0.17 mm. In some embodiments, the wall thickness WT is from 0.17 mm to 0.2 mm. In some embodiments, the wall thickness WT is from 0.13 mm to 0.17 mm. In some embodiments, the wall thickness WT is from 0.14 mm to 0.16 mm. In some embodiments, the wall thickness WT is about 0.15 mm. In some embodiments, the wall thickness WT is 0.15 mm.

Continuing to refer to FIG. 3, the device 100 has an overall length LT as measured parallel to the longitudinal axis A and from the first end 112 of the fitting section 110 to the tip 152. In some embodiments, the overall length LT is from 7.0 mm to 20.0 mm. In some embodiments, the overall length LT is from 7.0 mm to 16.75 mm. In some embodiments, the overall length LT is from 7.0 mm to 13.5 mm. In some embodiments, the overall length LT is from 7.0 mm to 10.25 mm. In some embodiments, the overall length LT is from 10.25 mm to 20.0 mm. In some embodiments, the overall length LT is from 10.25 mm to 16.75 mm. In some embodiments, the overall length LT is from 10.25 mm to 13.5 mm. In some embodiments, the overall length LT is from 13.5 mm to 20.0 mm. In some embodiments, the overall length LT is from 13.5 mm to 16.75 mm. In some embodiments, the overall length LT is from 16.75 mm to 20.0 mm. In some embodiments, the overall length LT is from 14 mm to 20 mm. In some embodiments, the overall length LT is from 15 mm to 19 mm. In some embodiments, the overall length LT is from 16 mm to 18 mm. In some embodiments, the overall length LT is about 17 mm. In some embodiments, the overall length LT is between 17 mm and 17.3 mm. In some embodiments, the overall length LT is 17.16 mm.

Referring now to FIG. 2, the device has a length L2 as measured parallel to the longitudinal axis A and from the transition between the concave portions 146 and the parallel portions 148 to the tip 152. In some embodiments, the length L2 is determined by the length along the insertion axis of an insert for which the device 100 is designated for use. In some embodiments, the length L2 is equal to the length along the insertion axis of an insert for which the device 100 is designated for use. In some embodiments, the length L2 is approximately equal to the length along the insertion axis of an insert for which the device 100 is designated for use. In some embodiments, the length L2 is in a range between (1) equal to the length along the insertion axis of an insert for which the device 100 is designated for use, and (2) 0.1 millimeter greater than the length along the insertion axis of an insert for which the device 100 is designated for use. In some embodiments, the length L2 is from 2 mm to 6 mm. In some embodiments, the length L2 is from 2 mm to 5 mm. In some embodiments, the length L2 is from 2 mm to 4 mm. In some embodiments, the length L2 is from 2 mm to 3 mm. In some embodiments, the length L2 is from 3 mm to 6 mm. In some embodiments, the length L2 is from 3 mm to 5 mm. In some embodiments, the length L2 is from 3 mm to 4 mm. In some embodiments, the length L2 is from 4 mm to 6 mm. In some embodiments, the length L2 is from 4 mm to 5 mm. In some embodiments, the length L2 is from 5 mm to 6 mm. In some embodiments, the length L2 is from 5.1 mm to 5.5 mm. In some embodiments, the length L2 is from 5.2 mm to 5.4 mm. In some embodiments, the length L2 is about 5.3 mm. In some embodiments, the length L2 is 5.3 millimeters.

Continuing to refer to FIG. 2, the device 100 has a length LI as measured parallel to the longitudinal axis A and from the first end 112 of the fitting section 110 to the transition between the concave portions 146 and the parallel portions 148. In some embodiments, the length L1 is determined as a function of the overall length LT of the device 100 and the length L2. In some embodiments, the length L1 is the overall length LT minus the length L2. In some embodiments, the length L1 is 11.9 mm.

Continuing to refer to FIG. 2, the device has a length L3 as measured parallel to the longitudinal axis A and from the first end 112 of the conical portion to the transition between the planar face 142 and the convex portions 144. In some embodiments, the length L3 is selected in order to ensure that an insert that is being inserted through use of the device 100 is suitably installed. In some embodiments, the length L3 is at least 0.1 millimeter greater than the length of the “beak” of an insert to be inserted. In some embodiments, the length L3 is in a range from 1 millimeter to 14 millimeters. In some embodiments, the length L3 is in a range from 1 millimeter to 10.75 millimeters. In some embodiments, the length L3 is in a range from 1 millimeter to 7.5 millimeters. In some embodiments, the length L3 is in a range from 1 millimeter to 4.25 millimeters. In some embodiments, the length L3 is in a range from 4.25 millimeter to 14 millimeters. In some embodiments, the length L3 is in a range from 4.25 millimeter to 10.75 millimeters. In some embodiments, the length L3 is in a range from 4.25 millimeter to 7.5 millimeters. In some embodiments, the length L3 is in a range from 7.5 millimeter to 14 millimeters. In some embodiments, the length L3 is in a range from 7.5 millimeter to 10.75 millimeters. In some embodiments, the length L3 is in a range from 10.75 millimeter to 14 millimeters. In some embodiments, the length L3 is in a range from 9 millimeters to 10 millimeters. In some embodiments, the length L3 is in a range from 9.2 millimeters to 9.6 millimeters. In some embodiments, the length L3 is about 9.5 millimeters. In some embodiments, the length L3 is about 9.4 millimeters. In some embodiments, the length L4 is 9.4 millimeters.

Continuing to refer to FIG. 2, the device has a length L4 as measured parallel to the longitudinal axis A and from the transition between the planar face 142 and the convex portions 144 to the tip 152. In some embodiments, the length L4 is determined as a function of the overall length LT of the device 100 and the length L3. In some embodiments, the length L4 is the overall length LT minus the length L3. In some embodiments, the length L4 is 7.75 millimeters.

Continuing to refer to FIG. 2, the fitting section 110 defines an aperture angle α. In some embodiments, the aperture angle α depends on an external device (e.g., an IOL inserter) to which the device 100 is configured to connect. In some embodiments, the aperture angle α is from 3.2 degrees to 8.5 degrees. In some embodiments, the aperture angle α is from 3.2 degrees to 7.2 degrees. In some embodiments, the aperture angle α is from 3.2 degrees to 5.9 degrees. In some embodiments, the aperture angle α is from 3.2 degrees to 4.5 degrees. In some embodiments, the aperture angle α is from 4.5 degrees to 8.5 degrees. In some embodiments, the aperture angle α is from 4.5 degrees to 7.2 degrees. In some embodiments, the aperture angle a is from 4.5 degrees to 5.9 degrees. In some embodiments, the aperture angle α is from 5.9 degrees to 8.5 degrees. In some embodiments, the aperture angle α is from 5.9 degrees to 7.2 degrees. In some embodiments, the aperture angle α is from 7.2 degrees to 8.5 degrees. In some embodiments, the aperture angle α is from 5.75 degrees to 6.75 degrees. In some embodiments, the aperture angle α is from 6 degrees to 6.5 degrees. In some embodiments, the aperture angle a is about 6.25 degrees. In some embodiments, the aperture angle α is 6.24 degrees.

Referring now to FIG. 3, the first and second parallel portions 148 define a width W1 when viewed as shown in FIG. 3. In some embodiments, the width W1 may vary depending on the size of an insert to be inserted through use of the device 100. In some embodiments, the width W1 is sufficiently wide so as to shelter a full width (e.g., diameter) of an insert to be inserted through the use of the device 100 from contact with the conjunctiva when the insert unfolds. In some embodiments, the width W1 is 1.5 to 2.5 millimeters. In some embodiments, the width W1 is 1.5 to 2.25 millimeters. In some embodiments, the width W1 is 1.5 to 2 millimeters. In some embodiments, the width W1 is 1.5 to 1.75 millimeters. In some embodiments, the width W1 is 1.75 to 2.5 millimeters. In some embodiments, the width W1 is 1.75 to 2.25 millimeters. In some embodiments, the width W1 is 1.75 to 2 millimeters. In some embodiments, the width W1 is 2 to 2.5 millimeters. In some embodiments, the width W1 is 2 to 2.25 millimeters. In some embodiments, the width W1 is 2.25 to 2.5 millimeters. In some embodiments, the width W1 is between 1.7 and 2.1 millimeters. In some embodiments, the width W1 is between 1.8 and 2.0 millimeters. In some embodiments, the width W1 is about 1.9 millimeters. In some embodiments, the width W1 is 1.91 millimeters.

Referring now to FIG. 4, the protrusion 120 defines an inner diameter ID1. In some embodiments, the inner diameter ID1 may vary depending on the size of an insert to be inserted through use of the device 100. In some embodiments, the inner diameter IDI is 1.5 to 3.0 millimeters. In some embodiments, the inner diameter ID1 is 1.5 to 2.75 millimeters. In some embodiments, the inner diameter ID1 is 1.5 to 2.5 millimeters. In some embodiments, the inner diameter ID1 is 1.5 to 2.25 millimeters. In some embodiments, the inner diameter ID1 is 1.5 to 2 millimeters. In some embodiments, the inner diameter ID1 is 1.5 to 1.75 millimeters. In some embodiments, the inner diameter ID1 is 1.75 to 3.0 millimeters. In some embodiments, the inner diameter ID1 is 1.75 to 2.75 millimeters. In some embodiments, the inner diameter ID1 is 1.75 to 2.5 millimeters. In some embodiments, the inner diameter IDI is 1.75 to 2.25 millimeters. In some embodiments, the inner diameter ID1 is 1.75 to 2 millimeters. In some embodiments, the inner diameter ID1 is 2 to 3 millimeters. In some embodiments, the inner diameter ID1 is 2 to 2.75 millimeters. In some embodiments, the inner diameter ID1 is 2 to 2.5 millimeters. In some embodiments, the inner diameter IDI is 2 to 2.25 millimeters. In some embodiments, the inner diameter IDI is 2.25 to 3 millimeters. In some embodiments, the inner diameter IDI is 2.25 to 2.75 millimeters. In some embodiments, the inner diameter ID1 is 2.25 to 2.5 millimeters. In some embodiments, the inner diameter ID1 is 2.5 to 3 millimeters. In some embodiments, the inner diameter ID1 is 2.5 to 2.75 millimeters. In some embodiments, the inner diameter ID1 is 2.75 to 3 millimeters. In some embodiments, the inner diameter ID1 is 1.5 to 1.9 millimeters. In some embodiments, the inner diameter ID1 is 1.6 to 1.8 millimeters. In some embodiments, the inner diameter ID1 is about 1.7 millimeters. In some embodiments, the inner diameter ID1 is 1.70 millimeters.

Referring now to FIG. 8, the protrusion 120 defines an arc angle γ representing the portion of a full (i.e., circular) cylinder spanned by the protrusion 120. In some embodiments, the arc angle γ may vary depending on the size of an insert to be inserted through use of the device 100. In some embodiments, the arc angle γ is between 90 degrees and 150 degrees. In some embodiments, the arc angle γ is between 90 degrees and 135 degrees. In some embodiments, the arc angle γ is between 90 degrees and 120 degrees. In some embodiments, the arc angle γ is between 90 degrees and 105 degrees. In some embodiments, the arc angle γ is between 105 degrees and 150 degrees. In some embodiments, the arc angle γ is between 105 degrees and 135 degrees. In some embodiments, the arc angle γ is between 105 degrees and 120 degrees. In some embodiments, the arc angle γ is between 120 degrees and 150 degrees. In some embodiments, the arc angle γ is between 120 degrees and 135 degrees. In some embodiments, the arc angle γ is between 135 degrees and 150 degrees. In some embodiments, the arc angle γ is between 100 degrees and 120 degrees. In some embodiments, the arc angle γ is between 105 degrees and 115 degrees. In some embodiments, the arc angle γ is about 110degrees. In some embodiments, the arc angle γ is 110 degrees.

Referring now to FIG. 2, the protrusion 120 defines a height H as measured perpendicularly to the longitudinal axis A from the first and second parallel portions 148 to the top 102. Based on the cylindrical geometry of the protrusion 120, the height H is a function of the width W1 shown in FIG. 3 and the angle γ shown in FIG. 8. As discussed above, in some embodiments, the width W1 may vary depending on the size of an insert to be inserted through use of the device 100. Accordingly, through its dependence on the width W1, in some embodiments, the height H may also vary depending on the size of an insert to be inserted through use of the device 100. In some embodiments, the height H is from 0.1 millimeters to 1.5 millimeters. In some embodiments, the height H is from 0.1 millimeters to 1.15 millimeters. In some embodiments, the height H is from 0.1 millimeters to 0.8 millimeters. In some embodiments, the height H is from 0.1 millimeters to 0.45 millimeters. In some embodiments, the height H is from 0.45 millimeters to 1.5 millimeters. In some embodiments, the height H is from 0.45 millimeters to 1.15 millimeters. In some embodiments, the height H is from 0.45 millimeters to 0.8 millimeters. In some embodiments, the height H is from 0.8 millimeters to 1.5 millimeters. In some embodiments, the height H is from 0.8 millimeters to 1.15 millimeters. In some embodiments, the height H is from 1.15 millimeters to 1.5 millimeters. In some embodiments, the height H is from 0.5 millimeters to 0.9 millimeters. In some embodiments, the height H is from 0.6 millimeters to 0.8 millimeters. In some embodiments, the height H is about 0.7 millimeters. In some embodiments, the height H is 0.7 millimeters.

Continuing to refer to FIG. 2, the first and second beveled portions 150 define a bevel angle δ with respect to the longitudinal axis A when viewed from the side as shown in FIG. 2. In some embodiments, the bevel angle δ is the base grind angle for the needle tip of the device 100, i.e., is the base angle from which the grinding of the first and second beveled portions 150 are formed. In some embodiments, the portion of the material of the device 100 that will form the first and second beveled portions 150 is first rough processed to the bevel angle δ, and then is finely processed to form the edges of the first and second beveled portions 150. In some embodiments, the bevel angle δ is between 10 degrees and 14 degrees. In some embodiments, the bevel angle δ is between 11 degrees and 13 degrees. In some embodiments, the bevel angle δ is between 11.5 and 12.5 degrees. In some embodiments, the bevel angle δ is about 12 degrees. In some embodiments, the bevel angle δ is 12 degrees. In some embodiments, the planar face 142 is also aligned with the angle δ (i.e., is angled with respect to the longitudinal axis A by the angle δ).

Continuing to refer to FIG. 2, the first and second beveled portions 150 define a thickness T, as viewed from the side as shown in FIG. 2. In some embodiments, the thickness T varies along an insertion direction. In some embodiments, the thickness T is sufficiently thin so as to provide for smooth insertion into the eye. In some embodiments, the thickness T defines the amount of material removed as measured perpendicularly to the plane defined by the angle δ that defines the first and second beveled portions 150. In some embodiments, the thickness T is from 0.08 to 0.2 millimeters. In some embodiments, the thickness T is from 0.08 to 0.17 millimeters. In some embodiments, the thickness T is from 0.08 to 0.14 millimeters. In some embodiments, the thickness T is from 0.08 to 0.11 millimeters. In some embodiments, the thickness T is from 0.11 to 0.2 millimeters. In some embodiments, the thickness T is from 0.11 to 0.17 millimeters. In some embodiments, the thickness T is from 0.11 to 0.14 millimeters. In some embodiments, the thickness T is from 0.14 to 0.2 millimeters. In some embodiments, the thickness T is from 0.14 to 0.17 millimeters. In some embodiments, the thickness T is from 0.17 to 0.2 millimeters. In some embodiments, the thickness T is from 0.13 to 0.17 millimeters. In some embodiments, the thickness T is from 0.14 to 0.16 millimeters. In some embodiments, the thickness T is about 0.15 millimeters. In some embodiments, the thickness T is 0.15 millimeters.

Continuing to refer to FIG. 2, the tip 152 forms a lancet angle δ with respect to the bevel angle δ defining the first and second beveled portions 150. In some embodiments, the lancet angle δ is configured to provide for smooth insertion of the tip 152 into the tissue of the eye. In some embodiments, the lancet angle δ is between 8 degrees and 12 degrees. In some embodiments, the lancet angle δ is between 9 degrees and 11 degrees. In some embodiments, the lancet angle δ is between 9.5 degrees and 10.5 degrees. In some embodiments, the lancet angle δ is about 10 degrees. In some embodiments, the lancet angle δ is 10 degrees.

In some embodiments, the tip 152 and the first and second beveled portions 150 form a sharp tip that is suitable for penetrating the conjunctiva of a human eye. In some embodiments, the top 102 of the device 100 is suitably smooth and polished so as to slide within the eye and below the conjunctiva once the tip 152 has penetrated into the eye. In some embodiments, the bottom 104 of the device 100 is suitably polished so as to avoid contact with the sclera of the eye after the tip 152 has penetrated into the eye. In some embodiments, at least one portion of the outer surface of the device 100 (e.g., the surface facing away from the longitudinal axis A) is smooth to a level of Ra 3.2 μm or greater. In some embodiments, at least one portion of the inner surface of the device 100 (e.g., the surface facing toward the longitudinal axis A) is smooth to a level of Ra 3.2 μm or greater.

In some embodiments, the insertion portion 140 can be referred to as having a “duck bill” shape having a profile that is sufficiently sized so as to allow an insert to pass through the device 100 (e.g., into the device through the first end 112 of the fitting section 110 and out of the device through the insertion portion 140) and be inserted into the eye with minimal contact with the conjunctiva. In some embodiments, the length of the insertion portion 140 (e.g., as measured along the longitudinal axis A) may vary depending on the length of an insert that is to be inserted by use of the device 100. In some embodiments, the length of the insertion portion 140 is longer than a length of an insert by at least 0.5 mm. In some embodiments, the length of the insertion portion 140 is longer than a length of an insert by no more than 2.0 mm. In some embodiments, the length of the insertion portion 140 is longer than a length of an insert by an amount between 0.5 mm and 2.0 mm. In some embodiments, the width of the insertion portion 140 (e.g., as measured perpendicular to the longitudinal axis A in a viewing plane as shown in FIG. 4) may vary depending on the width of an insert that is to be inserted by use of the device 100. In some embodiments, the width of the insertion portion 140 may vary depending on an unfolded width of an insert that is to be inserted into the eye while folded. In some embodiments, the width of the insertion portion 140 is at least 60% of the unfolded width of the insert.

In some embodiments, the device 100 is used as follows. A user couples the device 100 to an IOL inserter (or other suitable device) by engaging the fitting section 110 to the IOL inserter. The device 100 is inserted into the eye by penetrating the conjunctiva of an eye of a patient with the tip 152 and sliding the tip 152 to a desired insertion location. As discussed above, smoothness of the exterior surfaces of the device 100 allows the device to be moved easily to the insertion location with minimal friction. Once the device 100 is properly positioned, the user deploys an insert (e.g., an insert providing for delayed release of an API) through the device 100, exiting the device 100 via the insertion portion 140, in a manner consistent with standard operation of the IOL inserter. Following deployment of the insert, the user withdraws the device 100 from the eye. In some embodiments, the device 100 is single-use and is disposed of following withdrawal from the eye. In some embodiments, the device 100 is reusable and can be used for subsequent insertion processes after suitable sterilization. The foregoing exemplary method presents an improvement over prior techniques for insertion of such inserts, as only one insertion is required, as opposed to techniques in which a practitioner first forms a pocket with a scalpel, and subsequently inserts the IOL inserter into the pocket to deploy the insert. Such a one-insertion process therefore presents a one-step process for implantation of an IOL insert, as opposed to the two-step process required by prior devices. Consequently, the exemplary embodiments will simplify and shorten the placement procedure, thereby increasing patients' willingness to undergo the procedure and in parallel increase its success rate.

While a number of embodiments of the present invention have been described, it is understood that these embodiments are illustrative only, and not restrictive, and that many modifications may become apparent to those of ordinary skill in the art. For example, all dimensions discussed herein are provided as examples only, and are intended to be illustrative and not restrictive.

What is claimed is:

1. A device, comprising:
a body having a first end, a second end opposite the first end, a top side extending from the first end to the second end, and a bottom side opposite the top side,
wherein the body comprises:
a first portion having a hollow and substantially conical shape about a longitudinal axis, the first portion extending from the first end of the body to a transition intermediate the first and second ends of the body, and
a second portion having a hollow and substantially cylindrical shape about the longitudinal axis, the second portion extending from the transition to the second end of the body,
wherein the first portion and the second portion define a passage having a first inner diameter at the first end and a second inner diameter at the second end, wherein the first inner diameter is larger than the second inner diameter,
wherein the first end is configured to engage an intra-ocular lens injector;
wherein the second end includes a pointed tip configured to penetrate tissue of an eye, and
wherein an insertion opening is formed in the bottom side of the body and extends from the second end of the body toward the first end of the body,
wherein the insertion opening is configured to allow to pass therethrough an implantable device received at the first end of the body from the intra-ocular lens injector that is engaged with the first end of the body for implantation of the implantable device in the eye,
wherein the insertion opening, when viewed from a lateral side of the body, is defined by:
first and second beveled portions extending from the top side of the body toward the longitudinal axis and from the pointed tip toward the first end of the body, wherein each of the first and second beveled portions is concave with respect to the longitudinal axis when viewed from the lateral side of the body, and wherein the first and second beveled portions are positioned within the second portion of the body;
first and second parallel portions extending from corresponding ones of the first and second beveled portions in a direction toward the first end of the body, wherein each of the first and second parallel portions is substantially parallel to the longitudinal axis when viewed from the lateral side of the body, and wherein the first and second beveled portions extend across the transition between the first and second portions of the body;
first and second concave portions extending from corresponding ones of the first and second parallel portions in a direction toward the first end of the body and toward and past the longitudinal axis of the body, wherein the first and second concave portions are concave with respect to the longitudinal axis when viewed from the lateral side of the body, and wherein the first and second concave portions are positioned within the first portion of the body; and
a planar face extending from the first and second concave portions in a direction toward the first end of the body and away from the longitudinal axis of the body, wherein the planar face is angled at a bevel angle that is an acute angle with respect to the longitudinal axis of the body when viewed from the lateral side of the body, and wherein the planar face is positioned within the first portion of the body.

2. The device of claim 1, wherein respective transitions between the first and second concave portions and the planar face are curved.

3. The device of claim 1, wherein the bevel angle is in a range of from 10 degrees to 14 degrees.

4. The device of claim 3, wherein the pointed tip is angled with respect to the planar face at a lancet angle that is in a range of from 8 degrees to 12 degrees.

5. The device of claim 1, wherein the first and second concave portions have a radius of curvature that is in a range of from 1 millimeter to 3 millimeters.

6. The device of claim 1, wherein the pointed tip defines a tip angle that is in a range of from 60 degrees to 68 degrees.

7. The device of claim 1, wherein the device is configured for use with an implantable device having an implantable device length, and wherein the insertion opening has a length along the longitudinal axis that is greater than the implantable device length by from 0.5 mm to 2 mm.

8. The device of claim 1, wherein the second portion of the body and the first and second parallel portions define an arc angle that is in a range of from 90 degrees to 150 degrees.

9. The device of claim 1, wherein the second portion of the body has an inner diameter that is in a range of from 1.5 mm to 3 mm.

10. The device of claim 1, wherein the first end of the body has an inner diameter that is in a range of from 2 mm to 5 mm.

11. The device of claim 1, wherein a length of the device along the longitudinal axis is in a range of from 7 mm to 20 mm.
